(19) United States
(12) Patent Application Publication
(10) Pub. No.: US 2021/0205176 A1
(43) Pub. Date: Jul. 8, 2021

(54) PRODUCING INGREDIENT DELIVERY DEVICES FOR RELEASE CONTROL

(71) Applicant: HEWLETT-PACKARD DEVELOPMENT COMPANY, L.P., Spring, TX (US)

(72) Inventors: Wei HUANG, Palo Alto, CA (US); Gary J. DISPOTO, Palo Alto, CA (US)

(73) Assignee: HEWLETT-PACKARD DEVELOPMENT COMPANY, L.P., Spring, TX (US)

(21) Appl. No.: 16/075,876

(22) PCT Filed: Apr. 28, 2017
(86) PCT No.: PCT/US2017/030198

§ 371 Date: Aug. 6, 2018

(51) Int. Cl. A61J 3/00; A61K 9/20; B33Y 10/00; B33Y 50/02; B33Y 80/00

(52) U.S. Cl. CPC A61J 3/005; A61K 9/2095; A61K 9/2063; A61K 9/2059; B33Y 80/00; A61K 9/205; B33Y 10/00; B33Y 50/02; A61K 9/2054

(57) ABSTRACT

In an example implementation, a method of producing an ingredient delivery device includes applying a layer of powder within a work space, selectively depositing a liquid active ingredient onto the powder layer where the liquid active ingredient is to function as a fusing agent, and applying fusing energy to the powder layer to control a release profile of the active ingredient upon ingestion of the ingredient delivery device by a user.

### BACKGROUND

Accurate delivery of ingredients such as drugs and nutrients within a user's body can improve the therapeutic and nutritional impact of such ingredients. Accurate delivery of such ingredients can involve, for example, delivering multiple different ingredients, delivering the ingredients over a desired period of time, delivering the ingredients in particular doses, delivering the ingredients in varying doses over time, and so on. Products that enable such accurate delivery can provide improved convenience for users and help to reduce overall costs for consumers by improving the effectiveness and safety of the ingredients. Such products can include, for example, pills or tablets to be ingested by a user, and implant devices to be placed on or within a particular location of a user's body.

### BRIEF DESCRIPTION OF THE DRAWINGS

Examples will now be described with reference to the accompanying drawings, in which:

FIG. 1 shows a cross-sectional view of an example 3D printing process in which ingredient delivery devices can be produced for controlled release of active ingredients;

FIG. 2 shows cross-sectional views of some examples of ingredient delivery devices formed in an example 3D printing process in which the release of an active ingredient can be controlled according to the geometry of the ingredient delivery device;

FIG. 3 shows a cross-sectional view of an example ingredient delivery device that comprises a system of mini-tablets formed in an example 3D printing process in which the release of active ingredients can be controlled according to individually formed structures of each mini-tablet;

FIG. 4 shows examples of ingredient delivery devices exhibiting different diffusion schemes;

FIG. 5 shows a perspective view of an example 3D printing system suitable for printing ingredient delivery devices for controlled release of active ingredients;

FIG. 6 shows a perspective view of an example 3D printing system in which example ingredient delivery devices have been printed;

FIGS. 7, 8, and 9 are flow diagrams showing example methods of producing ingredient delivery devices for release control.

Throughout the drawings, identical reference numbers designate similar, but not necessarily identical, elements.

### DETAILED DESCRIPTION

The diagnosis of medical conditions, illnesses, general health and fitness issues, and so on, can often lead to a one-size-fits-all approach to managing such conditions, illnesses, and issues. That is, similar diagnoses often lead to the same prescribed treatments and medications. However, while a certain condition or set of conditions may be associated with a particular diagnosis, there are many factors that should be considered when determining a plan for treating such conditions. Taking such factors into account can help achieve a more effective personalized treatment. Factors that can help determine more effective personalized treatments include, for example, biological differences between different individuals such as height, weight, age, and sex; differences in the living and working environments of different individuals; and, differences in lifestyles that may impact interactions with different treatments, such as how an individual's diet may interact with a particular drug or medicine being considered for treatment.

Providing effective treatments tailored to an individual's personal physical makeup, environment, lifestyle, and so on, often involves customizing an active ingredient consumption regimen that can deliver active pharmaceuticals (e.g., drugs) and other ingredients (e.g., nutritional supplements) in varying dosages, over varying time frames, and to varying physical locations throughout the body. Thus, a doctor may prescribe drugs in a manner to try and achieve a particular therapeutic drug level within the body, such as having a constant drug concentration level within the body. However, achieving such levels using drugs that are not formulated for a controlled release may not be possible. For example, instead of achieving a constant drug concentration level within the body, the result may be an initial concentrated burst of the drug, followed by a gradual decrease in drug concentration over time. The same notion may generally apply as well when multiple drugs are involved. For example, a doctor may prescribe multiple drugs to be taken at different times and in different concentrations in order to achieve particular therapeutic levels within the body for each of the drugs. Again, achieving such levels may not be possible using drugs not formulated to provide controlled release.

As used here, the phrase “active ingredient”, is generally intended to refer to any of a variety of active pharmaceutical ingredients, drugs, medications, nutrients, pH level modifiers, flavors, and/or other ingredients to be consumed or applied for the treatment of various medical, nutritional, and/or other health related conditions. These terms and phrases may be used interchangeably throughout this description. In addition, throughout this description “active ingredient” may be referred to in shorthand as simply “AI”.

Products have been developed to assist individuals in self-administering active ingredient treatment regimens. These products can include ingredient delivery devices such as tablets, pills, capsules, and implantable devices that provide mechanisms to enable modified release of active ingredients. Modified release of an active ingredient generally refers to a modification in how the active ingredient is to be released and absorbed into the bloodstream or surrounding tissue. By contrast, immediate release can refer to the release of an active ingredient all at once, in a single dose. Ingredient delivery devices that provide for the modified release of active ingredients can function using a variety of different delivery modes including, for example, the delivery of multiple different ingredients, the delivery of ingredients over a desired period of time, delivering the ingredients in particular doses, delivering the ingredients in varying doses over time, and so on. Thus, ingredient delivery devices can be designed to provide customized release profiles for temporal and dose controlled delivery of multiple active ingredients that are specifically tailored to the conditions and health factors of each individual.

Customizable ingredient delivery devices can help to alleviate the difficulties associated with keeping track of medications, timing medications, and taking the proper dosages of medications. Prior methods for producing such devices include, for example, tablet press machines, powder mixers, pharmaceutical milling machines, and granulation machines that enable the production of tablets in customizable sizes, shapes, colors, coatings, and so on. More recent methods for producing such devices include 3D printing methods that can provide greater customizations such as personalized drug dosing and complex drug release profiles. In some examples, 3D printing methods used for producing drug tablets can involve the use of liquid binders applied to powder-based substrates. In some cases, tablets produced by such methods can result in tablets having poor mechanical durability, poor control of release profiles, and so on. In some examples, such anomalies may be attributable to the process steps in the liquid binder-based 3D printing method.

Accordingly, some example methods described herein enable the production of ingredient delivery devices that provide for controlled release of active ingredients (AI), such as pharmaceuticals, nutritional supplements, colorants, flavors, smells, and so on. An example 3D (three-dimensional) printing process can perform layer-by-layer additive manufacturing to construct ingredient delivery devices such as tablets, pills, capsules, and implantable devices that provide controlled release profiles. Controlled release profiles can be customized to particular active ingredients as well as to particular characteristics of a user, such as a user's biological, environmental, and lifestyle factors.

In an example 3D printing process, ingredient delivery devices can be built up layer-by-layer through the selective deposition (e.g., jetting) of liquid solutions and application of fusing energy to successive layers of powder material. The liquid solutions can comprise fusing agents, detailing agents, inks, and other liquids that are jettable from an inkjet printhead. The liquid solutions can also comprise an active ingredient, or multiple active ingredients. For example, jettable liquid solutions can comprise a mixture of fusing agent and an active ingredient where the active ingredient comprises a solute and the fusing agent comprises a solvent. Fusing energy can be controllably applied to each powder layer to cause selective fusing and/or sintering of the powder material in areas where the fusing agent has been applied, while areas where detailing agents have been applied can inhibit fusing and/or sintering. The controlled deposition of a “fusing agent-active ingredient” solution (FA-AI solution) and application of fusing energy onto powder layers can produce an ingredient delivery device that achieves a designed release profile for the active ingredient upon ingestion of the ingredient delivery device by a user. The release profile can include, for example, the timing of release of an active ingredient and the dosage of active ingredient being released. In an example 3D printing process, a number of factors can be controlled and adjusted to vary both the timing and dosing of an active ingredient including, for example, the concentration of active ingredient within the fusing agent solution, the deposition pattern of the solution, and the controlled application of fusing energy to the powder layer.

In an example process, ink and other jettable liquids can function as an active ingredient transporter as well as functioning as fusing and detailing agents. In addition, in some examples jettable liquid active ingredients can also function as fusing agents. In an example process, biocompatible powder can serve as the material of the active ingredient carrier (excipient) as well as the controller of the active ingredient release profile. In some examples, release profiles can be controlled in a variety of ways, including the distribution of fusing agent droplets that comprise active ingredients, the geometry of the ingredient delivery device being printed (e.g., a drug tablet), the release properties of the solid powder material, the microstructure of the material and the ingredient delivery device, and so on. In some examples, active ingredients can be carried in the powder material as well as in the ink, or instead of in the ink.

In a particular example, a method of producing an ingredient delivery device includes applying a layer of powder within a work space, selectively depositing a liquid active ingredient onto the powder layer where the liquid active ingredient is to function as a fusing agent, and applying fusing energy to the powder layer to control a release profile of the active ingredient upon ingestion of the ingredient delivery device by a user. In some examples, the amount of fusing energy to be absorbed by powder layers of the ingredient delivery device can be adjusted to alter the release profile of the active ingredient.

In another example, a non-transitory machine-readable storage medium stores instructions that when executed by a processor of a 3D printer arranged to produce ingredient delivery devices, cause the 3D printer to apply layers of biocompatible powder material within a work space, and for each layer, selectively apply a liquid solution of fusing agent and active ingredient that corresponds to a release profile of the active ingredient. For each layer, an amount of fusing energy is applied that corresponds to the release profile of the active ingredient.

In another example, a method of producing an ingredient delivery device includes applying within a work space, a layer of powder comprising an inactive ingredient and an active ingredient. The method also includes selectively depositing a liquid fusing agent solution onto the powder layer, and applying fusing energy to the powder layer to control a release profile of the active ingredient upon ingestion of the ingredient delivery device by a user.

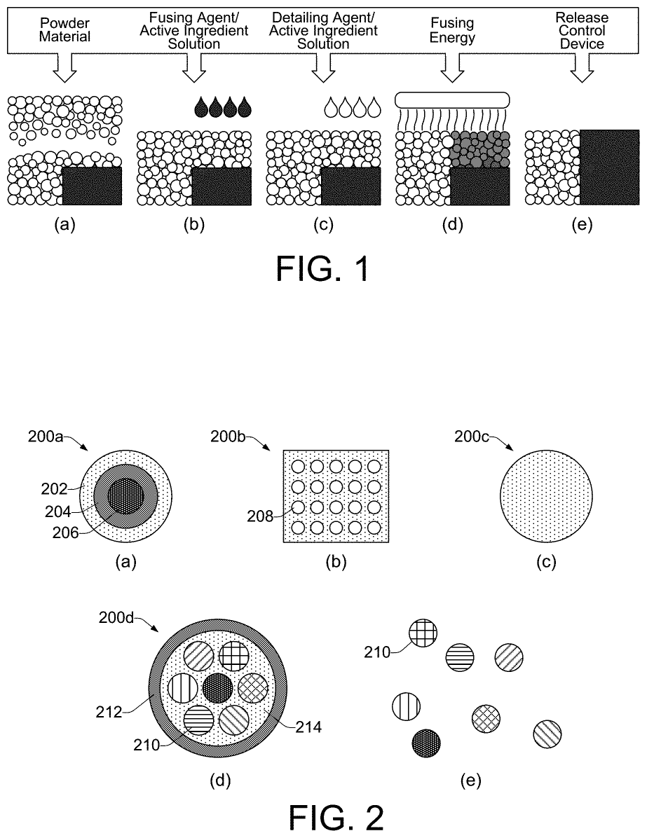

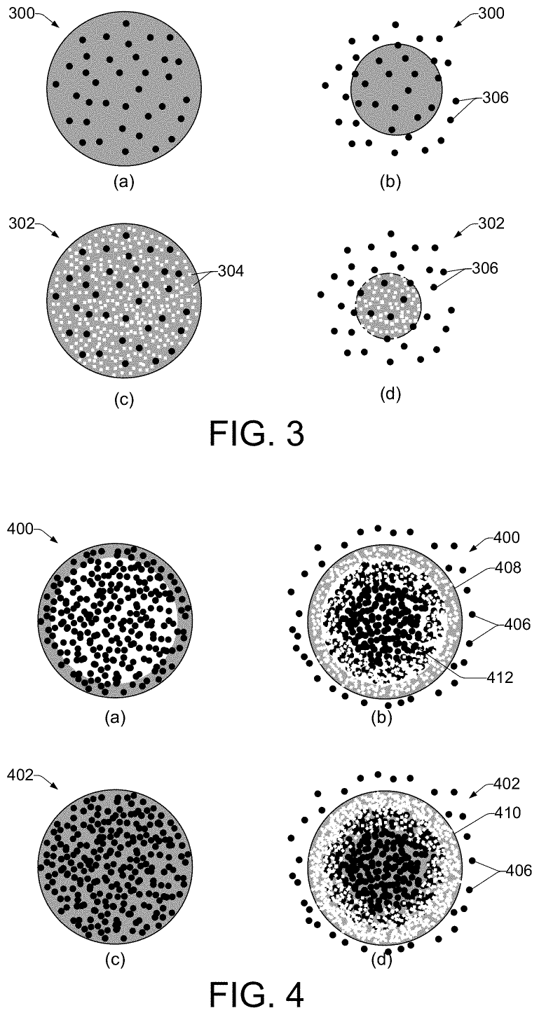

FIG. 1 shows a cross-sectional view of an example 3D printing process in which ingredient delivery devices can be produced for controlled release of active ingredients. In the example process, ingredient delivery devices can be produced layer-by-layer, through the selective deposition of liquid solutions and the application of fusing energy onto successive layers of powder material. FIG. 2 shows cross-sectional views of some examples of ingredient delivery devices formed in an example 3D printing process in which the release of an active ingredient, or multiple active ingredients, can be controlled according to the geometry of the ingredient delivery device. FIG. 3 shows a cross-sectional view of an example ingredient delivery device that comprises a system of mini-tablets formed in an example 3D printing process in which the release of active ingredients can be controlled according to individually formed structures of each mini-tablet.

Referring now generally to FIG. 1, in an example 3D printing process, a layer of powder material can be applied across a work space of a 3D printing device as shown in FIG. 1a. The work space can comprise, for example, the build platform of the device. The powder material can be applied over a previously applied powder layer (as shown in FIG. 1a), or directly onto the build platform of the work space when it is a first layer. The powder material can comprise a variety of inactive materials such as biocompatible materials that are ingestible and/or implantable materials, including for example, polymers, organics, gelatin, polysaccharides, carrageenans, starch, cellulose, flour, and combinations thereof. Some organic materials such as starch and flour can be fused when mixed with polymers due to the fusion of the polymers. In some examples, implantable materials can include metal and ceramic compositions in the powder material. Thus, the powder material can comprise an inactive substance that serves as an excipient carrier material to transport and deliver an active ingredient when ingested or implanted, for example. In some examples, the powder material can also comprise an active ingredient. Thus, the powder material may comprise a homogeneous mixture of inactive biocompatible powder material and an active ingredient in a powder form. Alternatively, or additionally, the powder material itself may be composed of an inactive biocompatible powder material and an active ingredient, such that each particle of the powder material consists of some ratio of inactive to active substance. In such examples where an active ingredient is included in the mixture or composition of the powder material, the liquid solution deposited onto the powder material may not include any active ingredient. That is, the liquid solution deposited onto the powder material may just be a fusing agent liquid solution.

As shown in FIG. 1b, a liquid solution can be selectively applied onto the powder layer where the particles of powder material are to be fused or sintered together. The liquid solution can comprise a fusing agent, an active ingredient, and/or a mixture of fusing agent and an active ingredient. In some examples, a fusing agent can be applied to the powder material separately, with or without a separate application of an active ingredient. In some examples, an active ingredient can be applied to the powder material separately, with or without a separate application of a fusing agent. In some examples, where an active ingredient is applied separately, without a fusing agent, the active ingredient can function both as an active ingredient and as a fusing agent, such as when an active ingredient is IR absorptive. A liquid solution comprising a mixture of one or multiple active ingredients (AI) as solute within a fusing agent (FA) as the solvent, may be referred to alternately herein as an “FA-AI solution”.

As shown in FIG. 1c, a liquid solution comprising a detailing agent can be selectively applied onto the powder layer where fusing of the powder material is to be reduced, prevented, or otherwise inhibited or altered. Detailing agents can include cooling agents and defusing agents, as discussed below. In some examples, a liquid solution comprising a mixture of detailing agent and an active ingredient can be selectively applied onto the powder layer. A liquid solution comprising a mixture of one or multiple active ingredients (AI) as solute within a detailing agent (DA) as the solvent, may be referred to alternately herein as a “DA-AI solution”. In general, the terms “fusing”, “fuse”, “fused”, and the like, indicate heating particles of the powder material to a level that involves fulling melting the particles to achieve solidification of the particles as a homogeneous part. The terms “sintering”, “sinter”, “sintered”, and the like, indicate heating particles of the powder material to a level that does not involve fulling melting the particles, but instead involves heating the particles of powder material to the point that the powder can fuse together on a molecular level. Thus, sintering generally enables control over the porosity of the material. However, because sintering involves a level of fusing particles together, the terms “fusing”, “fuse”, “fused”, may at times be used interchangeably with the terms “sintering”, “sinter”, “sintered”, depending on the context of the description. Thus, depending on the description, “fusing” may be used to indicate the solidification of particles of powder material that have not actually been fully melted, but instead have been partially melted. For example, in some instances a detailing agent can be deposited to reduce the fusing of particles within a particular area of powdered material in order to create porosity. In another example, an amount of fusing energy can be controlled (e.g., reduced) to a degree that particles of powdered material are partially melted rather than fully melted. Such actions may alternately be described as sintering, partial fusing, reduced fusing, and so on.

Referring generally to FIGS. 1b and 1c, fusing agents can comprise, for example, colored liquids such as carbon black ink that effectively target fusing energy (e.g., from an infrared light source) onto specific areas of the powder layer. Fusing agents can include water-based dispersions comprising a radiation absorbing agent such as an infrared light absorber, a near infrared light absorber, or a visible light absorber. Dye based and pigment based colored inks are examples of inks that include visible light absorbing agent. Darker fusing agents applied to the powder material generally cause a greater absorption of fusing energy into the powder, which causes higher temperatures and an increased melting and fusing together of the particles of powder. Detailing agents can comprise cooling agents and defusing agents. Detailing agents that comprise cooling agents can comprise, for example, liquids such as water that can cool the powder material during the application of fusing energy to prevent the powder from fully melting or fusing through controlling temperature. Detailing agents that comprise defusing agents can inhibit fusing chemically or mechanically. Such detailing agents can include other liquids such as silicon or oil that can be applied to mechanically and/or chemically inhibit fusing or sintering of the powder material.

As noted above, an active ingredient can include any of a variety of active pharmaceutical ingredients, drugs, medications, nutrients, and/or other ingredients to be consumed or applied for the treatment of various medical, nutritional, and/or other health related conditions. As shown in FIG. 1b, a liquid FA-AI solution comprising a mixture of one or multiple active ingredients within a fusing agent can be applied to the powder layer, for example, by jetting droplets of the liquid FA-AI solution through an inkjet printhead. Jetting the FA-AI solution enables precise placement of the fusing agent and the active ingredients onto the powder layer. The concentration of active ingredients within the FA-AI solution can be adjusted as one way to control dosing of the active ingredient. Likewise, a liquid DA-AI solution of detailing agent and active ingredients is also jettable to enable precise placement of the detailing agent and active ingredients onto the powder layer.

The selective application of FA-AI solution and DA-AI solution to each powder layer, along with the subsequent application of fusing energy, enables a layer-by-layer formation of the surface boundary of an ingredient delivery device, as well as the formation of the internal structure of the device. Thus, as each layer is fused, the boundary of the ingredient delivery device can take on a particular geometric shape, while the internal structure of the device can take on particular characteristics. Structural characteristics of the ingredient delivery device can be controlled through the selective application of fusing agents, detailing agents, and fusing energy (as discussed below). For example, the selective application of fusing agent and/or detailing agents enables the device to take on a variety of different structural characteristics, such as different porosities throughout the device, different levels of free or unfused powder material within the boundary of the device, and so on.

As shown in FIG. 1d, fusing energy can be applied to the powder layer after FA-AI solutions and DA-AI solutions have been applied. Fusing energy can be applied in a variety of ways, including for example, as infra-red (IR) radiation, near IR radiation, UV light, visible light emitting diodes (LEDs), lasers with specific wavelengths, heat lamps, and so on. Fusing energy can be controllably applied to each powder layer to control the level of fusing and/or sintering of the powder material. As noted above with regard to FIGS. 1b and 1c, the level of fusing and/or sintering of the powder material also depends on the fusing agents and detailing agents that may have been applied to the powder. Control over the level of fusing enables the creation of ingredient delivery devices with varying structural characteristics. Such characteristics can include, for example, the surface geometry of the delivery device, the internal porosity of the delivery device, the amount of unfused powder material free within the delivery device, and so on. Higher fusing energies, prolonged fusing exposure, and multiple fusing exposures, can increase the melting of powder material and thereby decrease the porosity of a delivery device. Lower fusing energies, reduced fusing exposure, and fewer fusing exposures, can reduce the melting of powder material and thereby increase the porosity of a delivery device. The amount of melting or sintering of powder material additionally depends on the amount and types of fusing and detailing agents that may be applied to the powder.

FIG. 1e shows an example of a portion of a layer of fused powder material, such as a layer of an ingredient delivery device. FIG. 2 shows cross-sectional views, or single layers, of some examples of ingredient delivery devices 200 (illustrated as devices 200a, 200b, 200c, 200d) that can be formed in an example 3D printing process as generally discussed above with regard to FIG. 1. The ingredient delivery devices 200 comprise active ingredient release profiles for releasing an active ingredient, or multiple active ingredients, that are controlled according to the geometry of the ingredient delivery device. Referring to FIG. 2a, for example, a multi-drug ingredient delivery device 200a has been produced with a release profile that is controlled by the relative geometric locations of the different active ingredients (drugs) within the delivery device 200a. More specifically, the active ingredients 202, 204, 206, have been arranged in an order from the outside to the inside of the delivery device 200a. Thus, the release of active ingredients 202, 204, 206, whether by erosion or diffusion, will occur from the outside to the inside of the device 200a.

Referring still to FIG. 2a, in an example 3D printing process, a first FA-AI solution comprising a first active ingredient 202 has been applied (i.e., jetted) onto the illustrated layer, or cross section, of the delivery device 200a at the outer boundary of the device 200a. On the same illustrated layer of the delivery device 200a, a second and different FA-AI solution comprising a second active ingredient 204 has been applied within, or inside, the area of the first active ingredient 202. A third FA-AI solution comprising a third active ingredient 206 has been applied in the center area of the illustrated layer of delivery device 200a. In some examples, a DA-AI solution may be applied to the illustrated layer of the delivery device 200a instead of or in addition to the active ingredients 202, 204, 206. A fusing energy applied to the illustrated layer of device 200a can then be controlled to fuse the areas 202, 204, 206 according to various factors including the intensity of the fusing energy, the time of exposure to the fusing energy, the number of exposures to the fusing energy, the types and amounts of fusing agent and detailing agent applied, and so on. The fusing can control the porosity, for example, of each area 202, 204, 206, of the device 200a. In some examples, the fusing can control the nature of the active ingredients stored or trapped within the areas 202, 204, 206, of the device 200a. For example, in some instances, greater or lesser fusing can be applied to control the state of the active ingredients. In different examples, active ingredients within an ingredient delivery device 200a can comprise solids, liquids, gases, solid-liquid combinations, solid-gas combinations, solid-liquid-gas combinations, and so on.

Referring to FIG. 2b, a layer or cross-section of an example ingredient delivery device 200b formed as a matrix structure is shown. Such a matrix structure provides a larger surface area for greater exposure of an active ingredient 208, which can result in a fast release profile. During an example 3D printing process, such a structure can comprise a homogeneous amount of active ingredient 208 filling the matrix. A homogeneous distribution of active ingredient can provide a release profile in which the rate of release of the active ingredient decreases with time, either through diffusion of the active ingredient, or through erosion of the device 200b. In some examples, an increasing amount of active ingredient 208 can fill the matrix, from the outside of the device to the inside of the device. An increasing distribution of active ingredient can provide a release profile in which the rate of release remains constant or increases.

Referring to FIG. 2c, a layer or cross-section of an example ingredient delivery device 200c is shown in which the device 200c provides a reduced surface area. The release profile of such an ingredient delivery device 200c can be slower than that of a matrix structure or other structure.

FIG. 2d shows a cross-sectional view of an example ingredient delivery device 200d that comprises a system of mini-tablets 210 formed in an example 3D printing process. An example delivery device 200d can provide multiple modified release profiles for different active ingredients associated with each mini-tablet 210. Different release profiles can include, for example, extended release, delayed release, pulsed release, binary release, and so on. In some examples, each mini-tablet 210 can comprise a distinct active ingredient. In some examples, during an example 3D printing process, the level of fusing applied to each mini-tablet 210, and thus the structure of each mini-tablet 210, can vary based on the types and amounts of applied fusing agents, detailing agents, and fusing energy.

Referring to FIG. 2d, during an example 3D printing process, a liquid fusing agent 212 without an active ingredient, can be applied onto a layer or cross-section of an example ingredient delivery device 200d. Upon fusing, the fusing agent area 212 can provide the boundary or outer surface of the ingredient delivery device 200d. Numerous different solutions of fusing agent and active ingredient (i.e., FA-AI solutions) can be deposited/jetted within the boundary area 212, and then fused with fusing energy to form the different mini-tablets 210. In some examples, the interior structure of the device 200d can comprise unfused or partially fused powder material 214 on which a detailing agent has been deposited. The ingredient delivery device 200d can release the mini-tablets 210 as shown in FIG. 2e, after which the unique release profiles of each mini-tablet 210 can control the release of a unique active ingredient.

As noted above, mechanisms for releasing active ingredients from an ingredient delivery device can include, for example, erosion and diffusion mechanisms. FIG. 3 shows examples of ingredient delivery devices exhibiting different erosion, or dissolution, schemes. In FIG. 3a, an ingredient delivery device 300 (e.g., a tablet) is shown prior to being dissolved. In FIG. 3b, the device 300 is in the process of eroding or dissolving. In FIG. 3c, a different ingredient delivery device 302 is shown prior to being dissolved. In FIG. 3d, the device 302 is in the process or erosion. The ingredient delivery device 300 has little or no porosity, while the ingredient delivery device 302 has an amount of porosity as indicated by the white, empty voids 304 evident in both FIGS. 3c and 3d. When comparing the progress of the erosion process between the partially dissolved devices of FIGS. 3b and 3d, it is apparent that the device 302 in FIG. 3d dissolves more quickly than the device 300 in FIG. 3b. Furthermore, the more porous device 302 of FIG. 3d releases its active ingredients 306 faster than the less porous device 300 of FIG. 3b. Thus, ingredient delivery devices that have greater amounts of porosity can dissolve more quickly and have a faster active ingredient release rate than similar ingredient delivery devices having less porosity. This is so, because devices with little or no porosity have limited surface area (i.e., the outer surface of the device) that comes in contact with water or other digestive fluids, while devices with greater porosity have greater surface area (i.e., the outer surface and the porous inner surface) that comes in contact with water or other digestive fluids, which results in faster dissolution and faster release rates.

Example 3D printing processes described herein comprise fusing operations that enable accurate control over the porosity of ingredient delivery devices through the control of various fusing related factors. Such fusing factors can include, for example, the amount of fusing energy applied to and absorbed by layers of powder material, the intensity or power of the fusing energy applied, the number of fusing applications or passes used, the duration of fusing applications, the type of fusing agents applied to the powder material, the type of detailing agents applied to the powder material, and so on. In general, higher levels of porosity are achieved with less fusing, such as when sintering occurs. Conversely, lower levels of porosity are achieved with increased fusing, such as when fusing causes the powder material to fully melt and fully fuse together. In some examples, free powder material that has experienced no fusing can have on the order of 50% porosity, while partially fused powder (i.e., sintered powder) can have on the order of 10% porosity, and fully fused powder that has been fully melted can have 0% porosity. Accordingly, the use of fusing in example 3D printing processes described herein to accurately control the porosity of ingredient delivery devices enables control over the release profiles of active ingredients.

FIG. 4 shows examples of ingredient delivery devices 400, 402, exhibiting different diffusion schemes. Such diffusion schemes are useful, for example, with implantable ingredient delivery devices. FIGS. 4a and 4b show an ingredient delivery device 400 with a constant barrier 408, while FIGS. 4c and 4d show an ingredient delivery device 402 with an increasing barrier 410. The structure of an ingredient delivery device 400, 402, such as barrier 408, does not dissolve or erode within the time frame in which the active ingredient 406 is released from the ingredient delivery device. Thus, active ingredients 406 are released from such delivery devices 400, 402, long before the devices will begin to dissolve. Diffusion of active ingredients from a non-dissolvable ingredient delivery device 400, 402, can occur when water or other fluids enter the active ingredient reservoir 412 of the device and contact the active ingredient, causing the active ingredient to dissolve into the fluid and/or be carried out of the delivery device. The device reservoir 412 can comprise free, unfused powder material, and/or porous material that has been partially fused, or sintered. As noted above, example 3D printing processes described herein comprise fusing operations that enable accurate control over the porosity of ingredient delivery devices through the control of various fusing related factors.

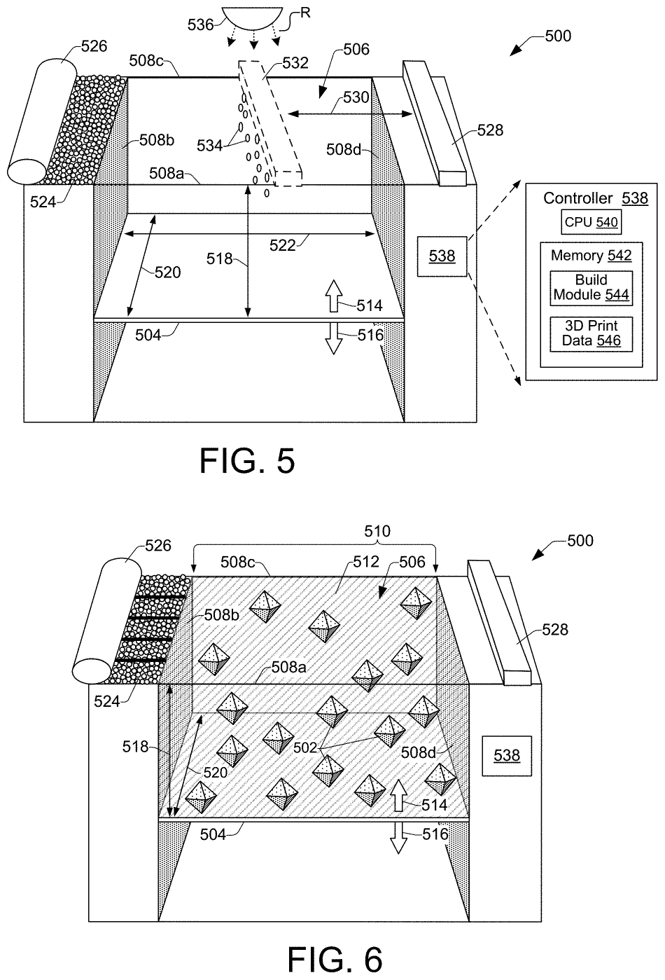

FIG. 5 shows a perspective view of an example of a 3D printing system 500 suitable for printing ingredient delivery devices 502 (e.g., tablets, pills, implants) for controlled release of active ingredients according to examples described herein. FIG. 6 shows a perspective view of an example of the 3D printing system 500 in which example ingredient delivery devices 502 have been printed. Referring to FIGS. 5 and 6, the 3D printing system 500 includes a moveable printing platform 504, or build platform 504. The printing platform 504 can serve as the floor to a work space 506 in which ingredient delivery devices 502 can be printed. The work space 506 can include fixed walls 508 (illustrated as front wall 508a, side wall 508b, back wall 508c, side wall 508d) that border the printing platform 504. The fixed walls 508 can contain a build volume 510 (FIG. 2a) comprising powdered build material that is deposited into the work space 506 during printing of an ingredient delivery device 502. During printing, the build volume 510 can include all or part of one or a number of ingredient delivery devices 502 either completed or partially completed in which powder layers have had fusing agents, active ingredients (i.e., FA-AI solutions), and fusing energy applied. The build volume 510 can also include non-processed powder material that surrounds the completed or partially completed ingredient delivery devices 502. Non-processed powder material can comprise a volume of reclaimable powder material 512 (illustrated in FIG. 6 as lightly shaded lines). For purposes of this discussion and to help illustrate different elements and functions of the 3D printing system 500, the front wall 508a of the work space 506 is shown as being transparent.

The printing platform 504 is moveable within the work space 506 in an upward and downward direction as indicated by up arrow 514 and down arrow 516, respectively. When the printing of ingredient delivery devices 502 begins, the printing platform 504 can be located in an upward position toward the top of the work space 506 as a first layer of powdered material is deposited onto the printing platform 504 and processed, for example, by applying fusing agents, detailing agents, active ingredients, and fusing energy. After a first layer of powder material has been processed, the printing platform 504 can move in a downward direction 516 as additional layers of powdered material are deposited onto the platform 504 and processed. Thus, the printing platform 504 can increase the height 518 dimension of the work space 506 to accommodate the production of additional ingredient delivery devices 502 by continuing to move downward 516. While the height 518 of the work space 506 is adjustable by movement of the printing platform 504 in a vertical direction, the depth 520 and width 522 dimensions of the work space 506 are fixed by the horizontal dimensions of the platform within the fixed walls 508.

Referring still to FIGS. 5 and 6, the example 3D printing system 500 includes a supply 524 of powdered material, or powder. As noted above, the powder material, alternately referred to herein as “powder”, can comprise inactive biocompatible powder materials such as polymers, organics, gelatin, polysaccharides, carrageenans, starch, cellulose, flour, and combinations thereof, that comprise ingestible and/or implantable materials. In some examples, the powder material in a powder supply 524 can also comprise an active ingredient. In some examples, a 3D printing system 500 can comprise multiple powder supplies 524 that contain different types of powder materials, and/or different mixtures of inactive biocompatible powder material and active ingredients. Powder materials from a supply 524 serve as the material of the ingredient delivery devices 502 which comprise active ingredient excipients-carriers. The 3D printing system 500 can feed powder material from a supply 524 into the work space 506 using a powder spreader 526 to controllably spread the powder into layers over the printing platform 504, and/or over other previously deposited layers of powder. In different examples, a powder spreader 526 can include a roller, a blade, or another type of material spreading device.

The example 3D printing system 500 also includes a liquid solution dispenser 528. While other types of liquid solution dispensers are possible, the example dispenser 528 shown and described herein comprises a printhead 528 or printheads, such as thermal inkjet or piezoelectric inkjet printheads. The example printhead 528 comprises a drop-on-demand printhead having an array of liquid ejection nozzles suitable to selectively deliver a solution of fusing agent and active ingredient (i.e., FA-AI solution), or other liquid, onto a layer of powder that has been spread onto the printing platform 504. In some examples, the printhead 528 has a length dimension that enables it to span the depth 520 of the work space 506 in a page-wide array arrangement as it scans over the work space 506 to apply droplets of FA-AI solution onto layers of powder within the work space 506. In FIG. 5, an example scanning motion 530 of the printhead 528 (shown by dashed-line printhead representation 532) is illustrated by direction arrow 530 as the printhead 528 scans across the work space 506 and ejects droplets of FA-AI solution 534 into the work space 506. Although not shown in the example of FIG. 5, in an actual printing scenario portions of ingredient delivery devices 502 would be present within the work space 506 as the printhead 528 scans over the work space and ejects droplets of the FA-AI solution 534, such as the ingredient delivery devices 502 shown in FIG. 6.

As shown in FIG. 5, the example 3D printing system 500 also includes a fusing energy source such as radiation source 536 (not shown in FIG. 6). The radiation source 536 can apply radiation R to layers of powder material in the work space 506 to facilitate the heating and fusing of the powder. A FA-AI solution 534 can be selectively applied by printhead 528 to a layer of powder material to enhance the absorption of the radiation R and convert the absorbed radiation into thermal energy, which can elevate the temperature of the powder sufficiently to cause curing (e.g., fusing, melting, sintering) of the particles of the powder. The radiation source 536 can be implemented in a variety of ways including, for example, as a curing lamp or as light emitting diodes (LEDs) to emit IR, near-IR, UV, or visible light, or as lasers with specific wavelengths. The radiation source 536 can depend in part on the type of fusing agent and/or powder being used in the printing process. The radiation source 536 can be attached to a carriage (not shown) and can be stationary or scanned across the work space 506.

The example 3D printing system 500 additionally includes an example controller 538. The controller 538 can control various operations of the printing system 500 to facilitate the printing of ingredient delivery devices 502 as generally described above, such as selectively applying fusing agent and active ingredient solutions (FA-AI solutions) to powder material layers in the work space 506, selectively applying detailing agent and active ingredient solutions (DA-AI solutions) to powder material layers in the work space 506, and controlling the application of fusing energy to the powder material layers. As noted above, controlling the fusing energy can include controlling the intensity of the fusing energy, the length of time the powder layer is exposed to the fusing energy, the number of exposures of fusing energy applied to a layer of powder material, and so on. Controlling the fusing energy enables the printing of ingredient delivery devices 502 with customized structures that provide controlled release profiles for the release of active ingredients.

As shown in FIG. 5, an example controller 538 can include a processor (CPU) 540 and a memory 542. The controller 538 may additionally include other electronics (not shown) for communicating with and controlling various components of the 3D printing system 500. Such other electronics can include, for example, discrete electronic components and/or an ASIC (application specific integrated circuit). Memory 542 can include both volatile (i.e., RAM) and nonvolatile memory components (e.g., ROM, hard disk, optical disc, CD-ROM, flash memory, etc.). The components of memory 542 comprise non-transitory, machine-readable (e.g., computer/processor-readable) media that can provide for the storage of machine-readable, executable, coded program instructions, data structures, program instruction modules, JDF (job definition format), 3D Manufacturing Format (3MF) print files, and other data and/or instructions executable by a processor 540 of the 3D printing system 500.

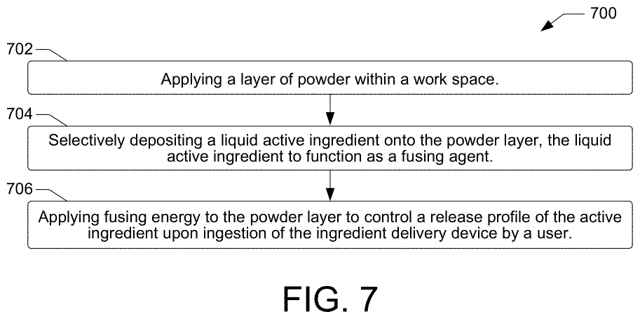

An example of executable instructions to be stored in memory 542 include instructions associated with a build module 544. Instructions from a build module 544 can be executable to control components of 3D printing system 500 to build ingredient delivery devices 502 according to data within a 3D print file 546. Thus, an example of stored data includes 3D print file data 546, alternately referred to as object data 546. In general, modules 544 and 546 include programming instructions and data executable by processor 540 to cause the 3D printing system 500 to perform operations related to printing ingredient delivery devices 502 within a work space 506, including controlling the application of fusing agents and fusing energy to control release profiles of the ingredient delivery devices 502. Such operations can include, for example, the operations of methods 700, 800, and 900, described below with respect to FIGS. 7, 8, and 9, respectively.

In some examples, controller 538 can receive 3D print file data 546 from a host system such as a computer. 3D print file data 546 can represent, for example, object files defining 3D ingredient delivery devices to be produced on the 3D printing system 500. Executing instructions from the build module 544, the processor 540 can generate print data for each cross-sectional slice of a 3D ingredient delivery device 3D print file data 546. The 3D print data 546 can define, for example, details for the application of fusing agents and active ingredients onto powder material layers, details for the application of fusing energy to powder material layers, and so on. The processor 540 can use the 3D print data 546 to control components of the printing system 500 to process each layer of powder material. Thus, the 3D print data 546 can be used to generate commands and/or command parameters for controlling the distribution of build powder material from a supply 524 onto the printing platform 504 by a spreader 526, the application of fusing agents by a printhead 528 onto layers of the powder, the application of fusing energy from a radiation source 536 to the layers of powder, and so on.

FIGS. 7, 8, and 9 are flow diagrams showing example methods 700, 800, and 900, of producing ingredient delivery devices for release control. Method 800 comprises extensions of method 700 that incorporate additional details. Methods 700, 800, and 900 are associated with examples discussed above with regard to FIGS. 1-6, and details of the operations shown in methods 700, 800, and 900 can be found in the related discussion of such examples. The operations of methods 700, 800, and 900 may be embodied as programming instructions stored on a non-transitory, machine-readable (e.g., computer/processor-readable) medium, such as memory 542 shown in FIG. 5. In some examples, implementing the operations of methods 700, 800, and 900 can be achieved by a processor, such as a processor 540 of FIG. 5, reading and executing the programming instructions stored in a memory 542. In some examples, implementing the operations of methods 700, 800, and 900 can be achieved using an ASIC and/or other hardware components alone or in combination with programming instructions executable by a processor 540.

The methods 700, 800, and 900 may include more than one implementation, and different implementations of methods 700, 800, and 900 may not employ every operation presented in the respective flow diagrams of FIGS. 7, 8, and 9. Therefore, while the operations of methods 700, 800, and 900 are presented in a particular order within their respective flow diagrams, the order of their presentations is not intended to be a limitation as to the order in which the operations may actually be implemented, or as to whether all of the operations may be implemented. For example, one implementation of method 800 might be achieved through the performance of a number of initial operations, without performing one or more subsequent operations, while another implementation of method 800 might be achieved through the performance of all of the operations.

Referring now to the flow diagram of FIG. 7, an example method 700 of producing ingredient delivery devices for release control begins at block 702 with applying a layer of powder within a work space. The work space can comprise, for example, a printing bed of a 3D printing system. As shown at block 704, the method 700 can include selectively depositing a liquid active ingredient onto the powder layer, where the liquid active ingredient is to function as a fusing agent. Fusing energy can then be applied to the powder layer to control a release profile of the active ingredient upon ingestion of the ingredient delivery device by a user, as shown at block 706.

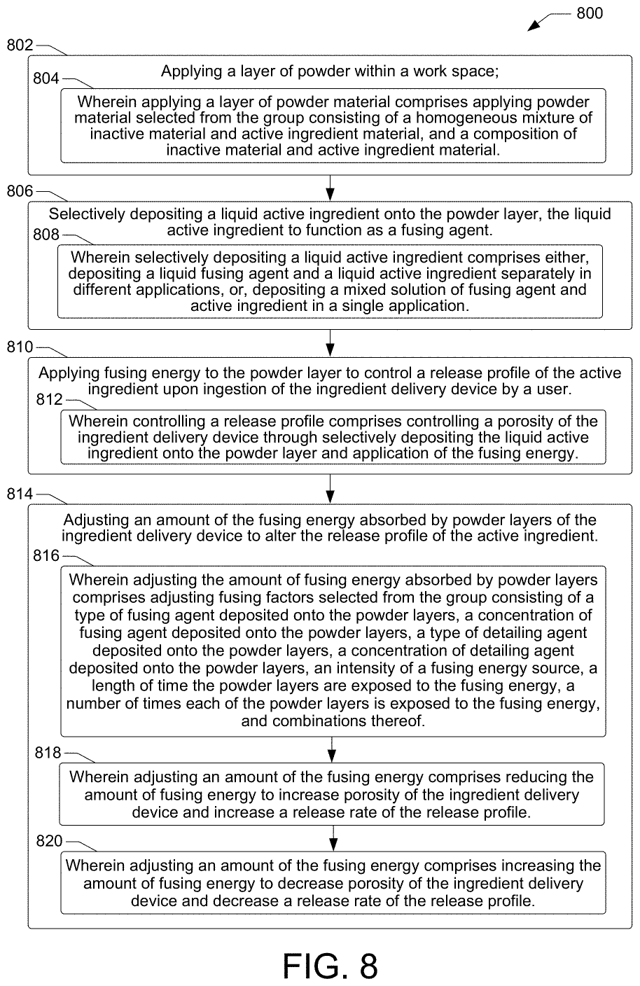

Referring now to the flow diagram of FIG. 8, another example method 800 of producing ingredient delivery devices for release control is shown. As noted above, method 800 comprises extensions of method 700 that incorporate additional details. Thus, method 800 can begin at block 802 with applying a layer of powder within a work space. In some examples, as shown at block 804, applying a layer of powder material comprises applying powder material selected from the group consisting of a homogeneous mixture of inactive material and active ingredient material, and a composition of inactive material and active ingredient material.

The method 800 can continue with selectively depositing a liquid active ingredient onto the powder layer, where the liquid active ingredient is to function as a fusing agent, as shown at block 806. In some examples, selectively depositing a liquid active ingredient onto the powder layer can include selectively depositing a fusing agent and active ingredient solution onto the powder layer, as shown in block 808.

The method 800 can include applying fusing energy to the powder layer to control a release profile of the active ingredient upon ingestion of the ingredient delivery device by a user, as shown at block 810. In some examples, controlling a release profile can include controlling a porosity of the ingredient delivery device through selectively depositing the liquid active ingredient onto the powder layer and through the application of the fusing energy, as shown at block 812. As shown at block 814, the method 800 can also include adjusting an amount of the fusing energy absorbed by powder layers of the ingredient delivery device to alter the release profile of the active ingredient. In some examples, as shown at block 816, adjusting the amount of fusing energy absorbed by powder layers comprises adjusting fusing factors selected from the group of factors consisting of a type of fusing agent deposited onto the powder layers, a concentration of fusing agent deposited onto the powder layers, a type of detailing agent deposited onto the powder layers, a concentration of detailing agent deposited onto the powder layers, an intensity of a fusing energy source, a length of time the powder layers are exposed to the fusing energy, a number of times each of the powder layers is exposed to the fusing energy, and combinations thereof. Adjusting an amount of the fusing energy can also include reducing the amount of fusing energy to increase porosity of the ingredient delivery device and increase a release rate of the release profile, as shown at block 818, and increasing the amount of fusing energy to decrease porosity of the ingredient delivery device and decrease a release rate of the release profile, as shown at block 820.

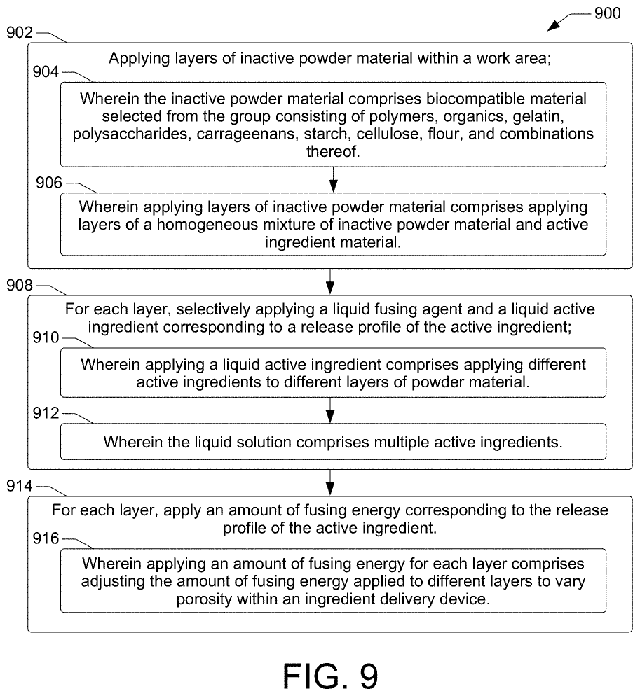

Referring now to the flow diagram of FIG. 9, another example method 900 of producing ingredient delivery devices for release control can begin at block 902 with applying layers of inactive powder material within a work area. As shown at block 904, in some examples the inactive powder material can comprise inactive biocompatible powder material selected from the group consisting of polymers, organics, gelatin, polysaccharides, carrageenans, starch, cellulose, flour, and combinations thereof. In some examples, applying layers of inactive powder material comprises applying layers of a homogeneous mixture of inactive powder material and active ingredient material, as shown at block 906. As shown at block 908, for each layer a liquid fusing agent and liquid active ingredient corresponding to a release profile of the active ingredient can be selectively applied. In some examples, as shown at block 910, applying a liquid active ingredient comprises applying different active ingredients to different layers of powder material. In some examples, the liquid solution comprises multiple active ingredients, as shown at block 912. As shown at block 914, an amount of fusing energy corresponding to the release profile of the active ingredient can be applied for each layer. In some examples, applying an amount of fusing energy for each layer includes adjusting the amount of fusing energy applied to different layers to vary porosity within an ingredient delivery device, as shown at block 916.

What is claimed is:

1. A method of producing an ingredient delivery device comprising:
applying a layer of powder within a work space;
selectively depositing a liquid active ingredient onto the powder layer, the liquid active ingredient to function as a fusing agent; and,
applying fusing energy to the powder layer to control a release profile of the active ingredient upon ingestion of the ingredient delivery device by a user.

2. A method as in claim 1, further comprising adjusting an amount of the fusing energy absorbed by powder layers of the ingredient delivery device to alter the release profile of the active ingredient.

3. A method as in claim 1, wherein selectively depositing a liquid active ingredient comprises either, depositing a liquid fusing agent and a liquid active ingredient separately in different applications, or, depositing a mixed solution of fusing agent and active ingredient in a single application.

4. A method as in claim 3, wherein adjusting the amount of fusing energy absorbed by powder layers comprises adjusting fusing factors selected from the group consisting of a type of fusing agent deposited onto the powder layers, a concentration of fusing agent deposited onto the powder layers, a type of detailing agent deposited onto the powder layers, a concentration of detailing agent deposited onto the powder layers, an intensity of a fusing energy source, a length of time the powder layers are exposed to the fusing energy, a number of times each of the powder layers is exposed to the fusing energy, and combinations thereof.

5. A method as in claim 1, wherein controlling a release profile comprises controlling a porosity of the ingredient delivery device through selectively depositing the liquid active ingredient onto the powder layer and through the application of the fusing energy.

6. A method as in claim 2, wherein adjusting an amount of the fusing energy comprises reducing the amount of fusing energy to increase porosity of the ingredient delivery device and increase a release rate of the release profile.

7. A method as in claim 2, wherein adjusting an amount of the fusing energy comprises increasing the amount of fusing energy to decrease porosity of the ingredient delivery device and decrease a release rate of the release profile.

8. A method as in claim 1, wherein applying a layer of powder material comprises applying powder material selected from the group consisting of a homogeneous mixture of inactive material and active ingredient material, and a composition of inactive material and active ingredient material.

9. A non-transitory machine-readable storage medium storing instructions that when executed by a processor of a three-dimensional (3D) printer for producing ingredient delivery devices, cause the 3D printer to:
apply layers of inactive powder material within a work area;
for each layer, selectively apply a liquid fusing agent and a liquid active ingredient corresponding to a release profile of the active ingredient; and,
for each layer, apply an amount of fusing energy corresponding to the release profile of the active ingredient.

10. A medium as in claim 9, wherein the inactive powder material comprises biocompatible material selected from the group consisting of polymers, organics, gelatin, polysaccharides, carrageenans, starch, cellulose, flour, and combinations thereof.

11. A medium as in claim 9, wherein applying layers of inactive powder material comprises applying layers of a homogeneous mixture of inactive powder material and active ingredient material.

12. A medium as in claim 9, wherein applying a liquid active ingredient comprises applying different active ingredients to different layers of powder material.

13. A medium as in claim 9, wherein the liquid active ingredient comprises multiple active ingredients.

14. A medium as in claim 9, wherein applying an amount of fusing energy for each layer comprises adjusting the amount of fusing energy applied to different layers to vary porosity within an ingredient delivery device.

15. A method of producing an ingredient delivery device comprising:
applying within a work space, a layer of powder comprising an inactive ingredient and an active ingredient;
selectively depositing a liquid fusing agent solution onto the powder layer; and,
applying fusing energy to the powder layer to control a release profile of the active ingredient upon ingestion of the ingredient delivery device by a user.
